(19) United States
(12) Patent Application Publication
(10) Pub. No.: US 2006/0259018 A1
(43) Pub. Date: Nov. 16, 2006

(54) Device for holding a medical instrument

(76) Inventor: Alexander Shilkrut, Brooklyn, NY (US)

Correspondence Address: Alexander Shilkrut, 2061 East 27 Street, Brooklyn, NY, 11229, US

(21) Appl. No.: 11/116,854

(22) Filed: Apr. 28, 2005

(51) Int. Cl. A61B 17/00

(52) U.S. Cl. 606/1

(57) ABSTRACT

According to one aspect of the invention, a device for holding a medical instrument comprises: (i) a hose including a first engagement element, the hose being position adjustable without the use of pressurized fluid; and (ii) an instrument holder with a second engagement element, wherein said first and the second engaging elements form a single ball and socket joint.

### BACKGROUND OF THE INVENTION

The present invention relates to improvements in diagnostic and surgical procedures, and especially to flexible holders for the stabilization of laparoscopes or other endoscopic instruments or surgical retractors used during diagnostic or surgical procedures. More particularly the invention relates an improved holder clamp which may be situated near the operating or examination table for supporting an endoscope, a retractor or the like.

### TECHNICAL BACKGROUND

Laparoscopic surgery is a procedure in which surgical instruments and a viewing scope, for example, laparoscope, are inserted through respective small puncture wounds or incisions into the abdominal cavity of a patient. A small video camera is attached to the laparoscope and connected to a television monitor for viewing the procedure.

The instruments and the laparoscope are inserted through cannulae which are first inserted through the incisions. Cannulae are hollow tubes with gas valves. The cannulae are left in the puncture wounds throughout the procedure. The cannulae allow the instruments and scope to be removed and reinserted as necessary

To aid in visualizing the intra-abdominal structures, a gas is provided through one of the cannulae to raise the abdominal wall. Seals are required at the exit points of the scope and instruments to prevent the gas from escaping. The viewing laparoscope is inserted through a cannula, which is usually inserted through an incision made in the umbilicus. The scope is then directed toward the pelvis for pelvic surgery or toward the liver for gallbladder surgery.

Throughout the procedure it is necessary for the surgeon, assistant surgeon, or a scrub nurse to hold the laparoscope and direct it at the target of the surgery. The laparoscope is being constantly repositioned to obtain the best view. The process of repositioning laparoscope ties up one hand of the surgeon or assistant surgeon, if either holds the laparoscope. The scrub nurses also have other tasks to perform, and holding the laparoscope interferes with performing these tasks. Furthermore, it is difficult for the surgeon to direct others to position the laparoscope for the best view. As a result, when the surgeon does not hold the laparoscope, it is often misdirected.

The existing medical instrument holding devices are either mechanical devices, or complicated electro-mechanical devices that are guided and voice activated. The former are cumbersome and difficult to use, the latter are very expensive.

U.S. Pat. No. 4,876,404 discloses a flexible holder for a cystoscope. This holder is a device that includes a mechanical hose and a clamping mechanism for supporting the cystoscope in the desired position. However, the clamping does not allow fine mobility of cystoscope because the clamping mechanism is rigidly attached to the end of the post, allowing movement only along the two axes and frequently necessitating readjustment of the whole flexible post when change in position is required. Furthermore, this holder includes many parts, requiring assembly and disassembly between procedures for sterilization. The clamping mechanism itself comprises six different components, making the overall device difficult to assemble and expensive to manufacture. Finally, the clamping mechanism of this device utilizes a metal spring that may become loose during surgery, making device unusable.

An endoscope holder is available commercially from Baitella AG, of Zurich, Switzerland and is also described in U.S. Pat. No. 4,431,329. This endoscope holder includes a sophisticated joint that secures adjoining arms by a turn of a knob. This endoscope holder does not allow for fine adjustments in the position of laparoscope, because its joints can not be individually adjusted. The master knob simultaneously regulates the position of three joints. When the master knob is in the “loose” position, all joints become loose, thus making the endoscope holder unstable and requiring repositioning of the whole device. Thus, all of the arms have to be repositioned and the master knob has to be tightened again. The joints can not be adjusted individually. For example, when the position of the laparascope needs to be adjusted slightly during the surgery, the whole endoscope holder needs to be re-adjusted. Finally, this endoscope holder is very complex and includes multiple interlocking parts that fit within one another. These parts require very precise manufacturing, contributing to the high cost of this device.

U.S. Pat. No. 5,447,149 discloses a complicated surgical instrument holder that comprises a flexible arm attached to an operating room table and has a clamping mechanism for holding a surgical instrument. This surgical instrument holder is relatively expensive to manufacture and includes many sophisticated components including a compressor for supplying pressurized fluid to pistons in order to provide joint reciprocal movement in response to pressure changes. Because this surgical instrument holder utilizes pressurized fluid, there is a possibility of fluid leak during the surgery that can result in electrical shorts.

U.S. Pat. No. 3,858,578 discloses a surgical retaining device that comprises a flexible arm (on one end of the device) attached to an operating room table with a clamping mechanism. The surgical instrument is attached into the other end of the flexile arm. This device is designed to hold a retractor (hook for holding tissues) and is unable to hold the laparoscope. In addition this device also requires fluid pressure for its operation.

Another manual device for holding a laparoscope is described in U.S. Pat. No. 4,573,452. This patent discloses that a rigid metal ring that surrounds the incision area is mounted above the surgical table. A vertical control arm is mounted on a ball-and-socket joint along the metal ring. A tensionable component connects the top of the control arm to a laparoscope holder. After the initial placement of the holder, the tensionable component is secured, after which movement of the laparoscope is achieved by pivoting the control arm about the ball and socket joint. It is suggested that the ball and socket joint be coplanar with the incision through which the laparoscope extends. The device disclosed in U.S. Pat. No. 4,573,452 requires the use of the ring, which is positioned over the patient. This ring can interfere with surgical procedures. In addition, a significant change in position of the laparoscope requires release and repositioning of the tensionable component.

As a result of complexity of use, inherent device limitations and high prices these medical instrument supporting devices are currently being underutilized. There is a need for device that will provide support for endoscopic or other medical instruments, and which is inexpensive and easy to utilize.

### SUMMARY OF THE INVENTION

According to one aspect of the invention, a device for holding a medical instrument comprises:
a hose including a first engagement element, the hose being position adjustable without the use of pressurized fluid; and
an instrument holder with a second engagement element, wherein said first and the second engaging elements form a single ball and socket joint.

According to one embodiment of the present invention the device for holding a medical instrument includes: (i) a base unit capable of attachment to an operating table including a flexible hose including the rod; and (ii) a stainless steel adjustable holder. The adjustable holder includes a housing and an instrument holder. The tightening of the housing against the rod of the hose (for example, by screwing the rod into the housing) forms a 360-degree swiveling mechanism (a ball and socket joint). The relative position of the housing with respect to the rod can be fastened and loosened, for example, by movement of the threaded surfaces of the rod and a housing relative to one another, which in turn allows for fastening or release of the joint, which will facilitate the positioning of medical instrument. Furthermore, the ball and socket of the joint may be in situated in a semi-locked position relative to one another, which will allow fine adjustments in the position of a medical instrument.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention, and together with the description serve to explain the principles and operations of the invention.

### BRIEF DESCRIPTION OF THE DRAWINGS

FIG. 1 is a perspective view of one embodiment of the device, according to the present invention;

FIG. 2 is a schematic cross section of a base unit of the device illustrated in FIG. 1

FIG. 3 is a schematic cross section of an adjustable holder of the device illustrated in FIG. I;

FIG. 4 is a perspective view of the adjustable holder illustrated in FIG. 3 with a second engagement element shown by dashed lines inside the holder;

FIG. 5 is a schematic cross section of device illustrating a ball and socket joint of the adjustable holder shown FIG. 4;

FIG. 6 is a perspective drawing of an alternative embodiment of an instrument holder; and

FIG. 7 is a cross-sectional view of an alternative instrument holder.

### DETAILED DESCRIPTION OF THE PREFERRED EMBODIMENTS

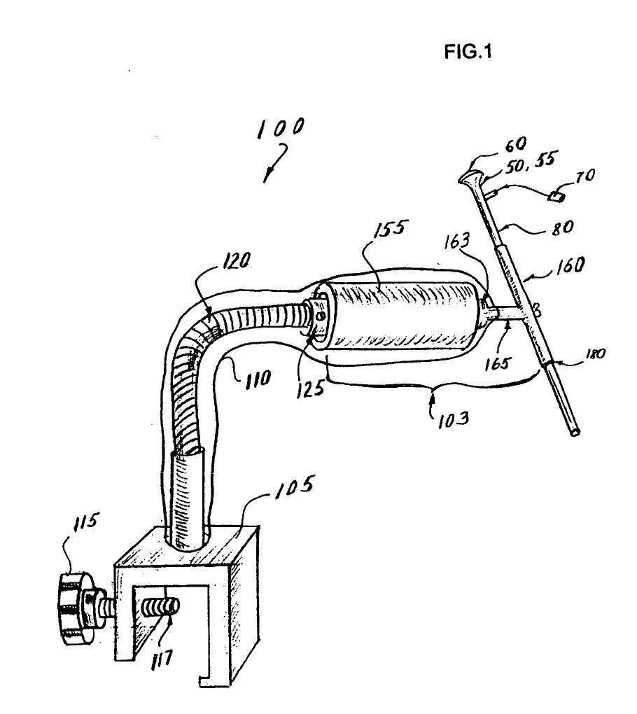

Reference will now be made in detail to the present preferred embodiment(s) of the invention, examples of which are illustrated in the accompanying drawings. Whenever possible, the same reference numerals will be used throughout the drawings to refer to the same or like parts. One embodiment of the present invention is shown in FIG. 1, and is designated generally throughout by the reference numeral 100.

More specifically, FIG. I shows a device 100 for holding a medical instrument 50. The medical instrument 50 is, for example, a typical laparoscopic camera 55 having an eyepiece 60, connector for the light source 70 and a sheath or shaft 80. The device 100 for holding a medical instrument includes a base unit 102 and an adjustable holder 103 that supports the medical instrument in the desired position and also allows 360° rotation and X-Y-Z motion of the medical instrument, as needed.

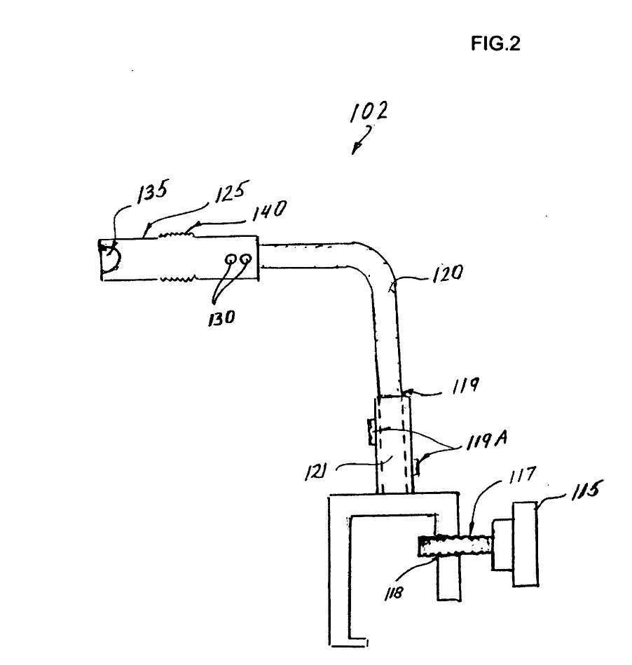

The base unit 102 is shown schematically in FIG. 2 and includes a clamp 105 for attachment to the operating room table or other surface. The clamp 105 is configured to embrace a rail of a standard operating room table and once positioned over the rail, it is securely fastened by a threaded screw 117, via a knob 115. The screw 117 is inserted through an aperture or an opening 118 in the clamp 105. The opening 118 has reciprocal threads for engaging with the screw 117. The clamp 105 may be removable. For example, if the rail has a non-standard size, the clamp 105 may be removed and replaced by another clamp 105 of the appropriate size. The clamp 105 also includes a shaft 119 that (i) engages a hose 120, and (ii) has at least one hose securing element 119A. The hose 120 has a rigid metal ending 121 (not shown) that engages the shaft 119 of the clamp 105 via the hose securing elements 119A, for example a screw or a bolt. In a case of non standard operating room table, by releasing hose securing element 119A, one will expose a metal ending 121 suitable for clamping to other commercially available clamps.

The clamp 105 may be made of steel or other material. The flexible hose 120 is equipped with a plurality of sections and precision ball joints, connected by internally positioned flexible cable, and also secured by plastic sheath wrapped around the hose. These hoses are commercially available from several vendors for example Rycor Medical Inc, Cleveland, Ohio. Also examples of similar flexible hoses are shown in U.S. Pat. Nos. 239,131 and 4,867,404, which are incorporated by reference herein. It should be understood that that the hose 120 is a part of a base unit that is not in direct contact with the patient, and that the hose could be manufactured form different plastic materials as long as it has enough flexibility to assume desired position.

The flexible hose 120 is attached to both the clamp 105 and a rod 125 that engages the adjustable holder 103. The adjustable holder 103 supports the medical instrument 50 in the fixed position and is capable of supporting it in any required position, as needed. Referring again to FIG. 2, the flexible hose 120 is attached to the rod 125, by means of rod securing elements 130, screws or bolts. The rod 125 is a tubular structure that is secured to the hose 120 and includes a first engaging element 135. The first engaging element 135 may be in the form of a concave or convex surface that forms a part of a flexible ball and socket joint 137. The rod 125 also has a threaded surface 140 that allows the adjustable holder 103 to be fastened against the rod so as to assume a desired position. The rod 125 is a part of the base unit illustrated in FIG. 2 and it may be made of a stainless steel. It also doesn't require sterilization between procedures.

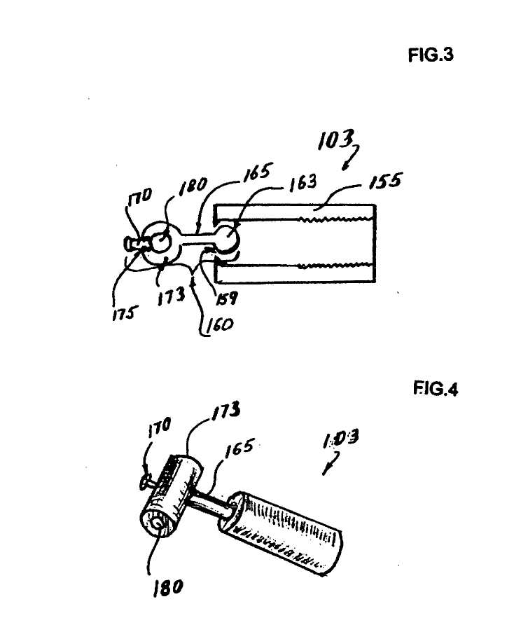

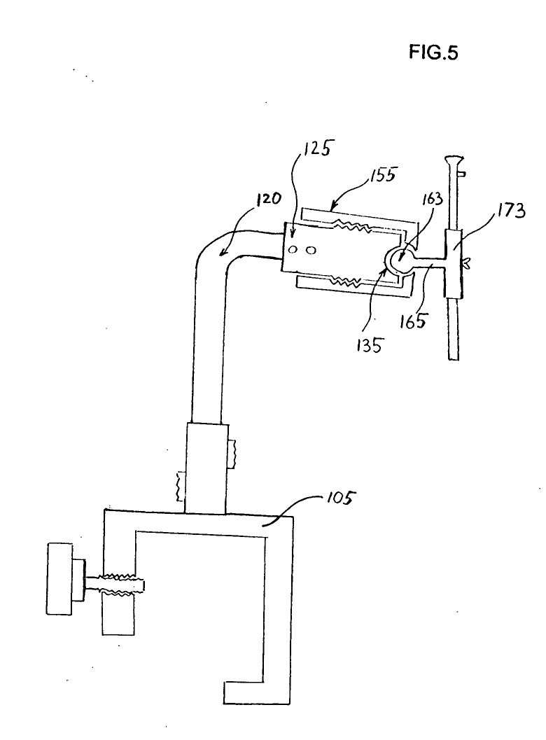

FIGS. 3 and 4 illustrate schematically the adjustable holder 103 for holding and positioning the medical instrument 50. The adjustable holder 103 includes housing 155 for the rod 125 and an instrument holder 160. The housing 155 shown in FIGS. 3 and 4 has internal threaded surface 157 that allows it to be securely screwed onto a rod 125. When the housing 155 is screwed tightly against the rod 125, the second engagement element 163 (pictured here as a ball 163A), comes into proximate contact with a first engagement element 135 (for example, concave surface of a rod 125), thus allowing secure and precise positioning of a medical instrument attached to an instrument holder 160. Alternatively the first engagement element could be a convex surface, while the second engagement element could be a concave surface that slidably engages the convex surface of the first engagement element and is attached to the finger 165. Other ways of engaging the housing 155 and the rod 125 can also be utilized.

When the housing 155 is screwed loosely on a threaded surface 140 of the rod 125, it allows for a space between the first and second engaging elements, respectively depictured as socket 135 and ball 163 connections. When there is a space between first and second engaging elements, it allows for flexibility and easy adjustment of medical instrument 50 along multiple axes. An instrument holding clamp 173 is attached to the second engagement element 163 via finger 165, protruding through an aperture 159 in the housing 155. FIG. 5 illustrates schematically the ball and socket joint of the adjustable holder shown FIG. 4. Thus, the adjustable holder includes a 360-degree swiveling mechanism formed by a ball and socket joint, and the relative position of the housing with respect to said rod can be fastened and loosened (for example, via the treaded movement of the rod relative to the housing), thus tightening or releasing said joint which, will facilitate the positioning of medical instrument. Furthermore, the housing 155 is adapted to serve as a handle for adjustment of the position of the hose relative to the operating room table. That is, the housing 155 is preferably cylindrical in shape and can be easily grasped and moved by a surgeon during the surgery, thereby adjusting the position of the hose 120. The ball an socket joint is capable of being in a semi-locked position, thereby allowing fine adjustments of the position of the medical instrument

FIGS. 3-4 present the configuration of an instrument holding clamp 173, which is comprised of a hollow cylinder. A medical instrument 50 is threaded through an aperture 180 in the instrument holding clamp 173 and secured by a bolt threaded through the opening 175. It should be understood that different aperture sizes can accommodate different instruments, and also the shape of aperture can be changed to accommodate commonly used instruments, such as laparoscopic camera, that usually contains cylindrical shaft 80 which is about 10 millimeters in diameter. The bolt 170 has the preset length, so that when fully tightened it protrudes only few millimeters into the aperture 180 of the instrument holding clamp 173, such that it prevents the movement of the instrument without damaging the shaft 80, as depicted in the FIG. 1.

The adjustable holder 103 is preferably made of stainless steel. It comes into direct contact with medical instrument, but not with the patient and preferably should be sterilized between the surgical procedures.

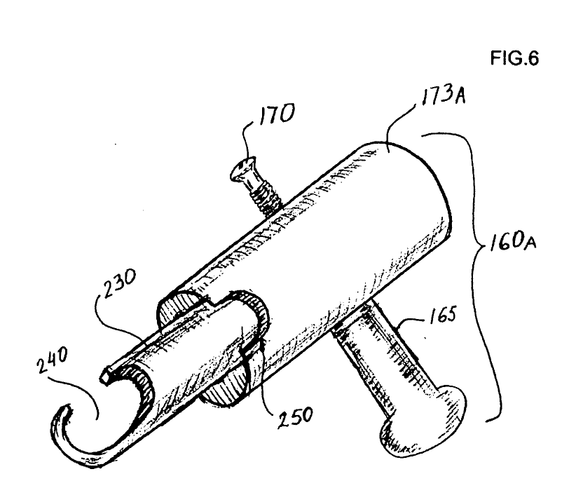

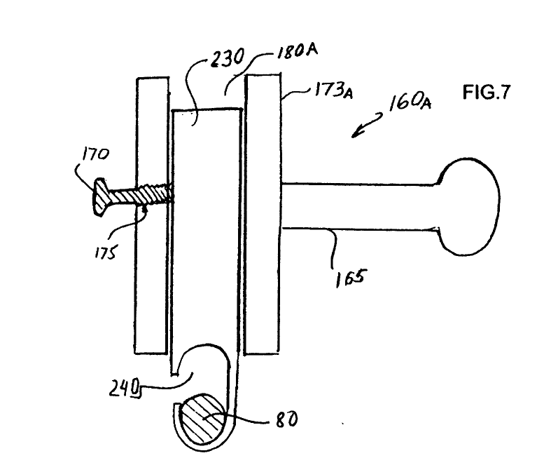

Alternatively, the instrument holder may have configuration illustrated in FIGS. 6 and 7. The alternative instrument holder 160_{A} includes a tubular instrument holding clamp 173_{A}, a finger 230 with a c-shaped opening 240, and a bolt 170. This instrument holding clamp 173_{A} is attached, via the finger 165, to the second engagement element 163 of device 100. The finger 230 is inserted into the instrument holding clamp 173_{A} and secured in the desired position by bolt 170. The bolt 170 may be threaded trough the opening 175 of the instrument holding clamp 173_{A}. The alternative instrument clamp 173_{A} is similar to the instrument holding clamp 173 of FIG. 3, except that it includes a concave surface 250. The finger 230 is placed inside the aperture 180_{A} of the instrument holding clamp 173_{A}, so the finger 230 slides easily inside the instrument holding clamp 173_{A}.

By sliding the finger 230 inside the instrument holding clamp 173_{A}, one can change the size of the opening 240 to accommodate a variety of medical instruments. Once the desired size opening is achieved, the finger 230 is secured inside the instrument holding clamp 173 A by the bolt 170. This embodiment maintains minimal pressure on the shaft 80 of medical instrument 50, and ensures gentle and secure holding of the medical instrument. The clamp includes of only few parts, is easy to manufacture, and its simplicity of design minimizes the chance of malfunctioning.

Thus, according to one embodiment of the present invention the device for holding a medical instrument includes: a base unit 102 capable of attachment to an operating table including a flexible hose 120 including the rod 125; and a stainless steel adjustable holder 103. The adjustable holder 103 includes a housing 155 and an instrument holder 160. The tightening of the housing 155 against the rod 125 forms a 360-degree swiveling mechanism (a ball and socket joint). The housing 155 screws on the rod 125, thus the relative position of the housing 155 with respect to the rod 125 can be fastened and loosened by the relative movement of the threaded surfaces of the rod and a housing, which in turn allows for fastening or releasing the joint which will facilitate the positioning of medical instrument 50. Also the ball and socket of the joint may be in located in a semi-locked position relative to one another which will allow fine adjustments in the position of a medical instrument.

During the procedure, a disposable plastic sheath 110, for example one available commercially from Microtek Medical, Inc is secured around the finger 165 and covers the housing 155, the hose 120 and the clamp 105, to allow the maintenance of sterile field during the procedure and eliminating the need for sterilization of the base unit 102 depictured in FIG. 2.

The base unit 102 doesn't require to be sterilized between surgical procedures because it covered by a sterile (for example plastic) sheath.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

What is claimed is:

1. A device for holding a medical instrument comprising:
(i) a hose including a first engagement element, said hose being position adjustable without the use of pressurized fluid; and
(ii) an instrument holder with a second engagement element, said first and second engaging elements forming a single ball and socket joint.

2. The device for holding a medical instrument according to claim 1 further comprising an adjustable holder secured to said hose and including said instrument holder.

3. The device for holding a medical instrument according to claim 2, wherein said adjustable holder adjustably secured to said hose.

4. The device for holding a medical instrument according to claim 2, wherein said hose includes a rod comprising said first engagement element, said adjustable holder including a housing adjustably secured to said rod.

5. The device for holding a medical instrument according to claim 4 wherein said instrument holder further includes a finger attached to said second engagement element and an instrument holding clamp.

6. The device for holding a medical instrument according to claim 4 wherein said housing includes an aperture for said finger.

7. The device for holding a medical instrument according to claim 4, wherein said housing and said rod are attached to one another via a threaded surface and the relative position of said housing with respect to said rod releases and immobilizes the instrument holder in the desired position.

8. The device for holding a medical instrument according to claim 4, wherein the housing is adapted to serve as a handle for adjustment of the position of the hose relative to the operating room table.

9. The device for holding a medical instrument according to claim 1, further including a clamp attached to said hose, said clamp being configured to be capable of attachment to a surgical table.

10. The device for holding a medical instrument according to claim 1, wherein said hose and at least a portion of said adjustable holder is wrapped in disposable sheath.

11. The device for holding a medical instrument comprising:
(iii) a base unit capable of attachment to an operating table including a flexible hose with a rod;
(iv) a stainless steel adjustable holder attached to said flexible hose and including (a) a housing and (b) an instrument holder and includes a 360-degree swiveling mechanism formed by a ball and socket joint at least partially located within said housing.

12. The device for holding a medical instrument according to claim 11 wherein the housing screws on said rod and the relative position of the housing with respect to said rod can be fastened and loosened, thus tightening or releasing said joint which will facilitate the positioning of medical instrument.

13. The device for holding a medical instrument according to claim 12, wherein said joint is capable of being in a semi-locked position, thereby allowing fine adjustments of the position of the medical instrument.

14. The device for holding a medical instrument according to claim 12, wherein said base unit includes a c-clamp attached to said hose, said c-clamp being configured so as to be capable of attachment to a surgical table.

15. The device for holding a medical instrument according to claim 11, wherein said hose and at least a portion of said adjustable holder are wrapped in a disposable sheath.

16. The device for holding a medical instrument comprising: a base unit capable of attachment to an operating table including a flexible hose wit a rod; and an adjustable holder including (a) a housing and (b) an instrument holder, such that said housing and said instrument holder are configured to form a 360-degree swiveling mechanism via a ball and socket joint; and said housing engages said rod such that said housing and said rod are capable of moving relative to each other.

17. The device for holding a medical instrument according to claim 1 wherein said adjustable holder includes a finger with a C-shape opening capable of receiving a medical instrument.

18. The device for holding a medical instrument according to claim 1 further comprising an instrument holding clamp.

19. The device for holding a medical instrument according to claim 18 further comprising an instrument holding clamp and a finger with a C-shape opening capable of receiving a medical instrument, wherein said finger is movable inside the instrument holding clamp.

20. The device for holding a medical instrument according to claim 16, further including a clamp attached to said hose, said clamp being configured to be capable of attachment to a surgical table.
